(19) United States
(12) Patent Application Publication
(10) Pub. No.: US 2023/0232186 A1
(43) Pub. Date: Jul. 20, 2023

(54) ASSET TRACKING TECHNOLOGIES

(71) Applicant: PatienTech LLC, Morning View, KY (US)

(72) Inventors: Robin Kjaldgaard, Winnipeg (CA); Andrew J. Frank, Winnipeg (CA); Robert Golden, Morning View, KY (US)

(21) Appl. No.: 17/998,132

(22) PCT Filed: May 7, 2021
(86) PCT No.: PCT/US2021/031362

§ 371 Date: Nov. 7, 2022

Related U.S. Application Data
(60) Provisional application No. 63/022,220, filed on May 8, 2020.

(51) Int. Cl. H04W 4/029; H04W 4/02; G16H 40/67; G08B 7/06

(52) U.S. Cl. CPC H04W 4/029; H04W 4/025; G16H 40/67; G08B 7/06

(57) ABSTRACT

Technologies for tracking the location of mobile assets include a tracking device mounted to an asset and radio-frequency identification tags installed or attached to static structures. The radio-frequency identification tags include identification data stored thereon. The identification data is associated with the installed location of the corresponding radio-frequency identification tags. The tracking device includes one or more transceivers configured to energize or trigger the radio-frequency identification tags and receive the stored identification data when the tracking device and asset are in proximity to the tags. The current location of the mobile asset is determined based on the identification data received from the radio-frequency identification tags.

### CROSS-REFERENCE TO RELATED APPLICATION

This applications claims the priority of U.S. Provisional Patent App. 63/022,220, filed May 8, 2020, and titled “Asset Tracking Technologies,” the entire disclosure of which is incorporated herein by reference.

### TECHNICAL FIELD

Embodiments of the technologies disclosed herein relate, in general, to asset tracking. More particularly, the technologies disclosed herein relate to systems, apparatuses, and methods for tracking the location and movement of physical assets within structures or other defined areas.

### BACKGROUND

Tracking the location and movement of physical assets or objects within structures or other defined areas is a persistent problem in many industries. For example, it is often difficult to accurately track the location and movement of physical assets such as beds, supply carts, infusion pumps, mobile computers, stands, and other types of equipment within hospital buildings or across medical campuses. It, however, is often necessary for medical personnel to quickly locate a particular asset, especially in emergency or other urgent situations. Many of these assets are expensive and, despite their size, they are subject to an increased risk of theft because of their value.

Conventional asset tracking techniques, such as the use of GPS tracking and/or conventional radio-frequency identification (“RFID”) technology are often susceptible to physical and/or electromagnetic interference and are therefore typically unable to reliably operate in large buildings, especially those having concrete and/or metal substructures. Additionally, such conventional techniques are typically ineffective over large distances and across campuses, between buildings, on different levels of a structure, or within other defined areas. Manual record-keeping techniques often result in inaccurate data being recorded (e.g., incorrect location or date and time recorded, etc.) or the recorded data is stale (e.g., an asset was moved after the data was recorded). Consequently, physical assets or objects—and in some cases objects or people associated therewith—are often misplaced and difficult to locate.

### BRIEF DESCRIPTION OF THE DRAWINGS

It is believed that certain embodiments will be better understood from the following description taken in conjunction with the accompanying drawings, in which like references indicate similar elements and in which:

FIG. 1 is an illustrative diagram of at least one embodiment of an asset tracking system;

FIG. 2 depicts a simplified system processing flow diagram of an embodiment of the asset tracking system of FIG. 1;

FIG. 3 is an illustrative diagram of at least one embodiment of an RFID transceiver that may be used with the asset tracking device of FIGS. 1 and 2;

FIG. 4 is an illustrative diagram of at least one embodiment of RFID tag placement within a doorway that may be used with the asset tracking system of FIGS. 1 and 2;

FIG. 5 is an illustrative diagram of at least one embodiment of an asset tracking system;

FIG. 6 is a simplified flowchart showing a set of steps that may be performed with an asset tracking system configure an asset for tracking;

FIG. 7 is a simplified flowchart showing a set of steps that may be performed with an asset tracking system to determine a location of a tracked asset; and

FIG. 8 is a simplified flowchart showing a set of steps that may be performed with an asset tracking system to provide location and motion based alerts.

### DETAILED DESCRIPTION

Various non-limiting embodiments of the present disclosure will now be described to provide an overall understanding of the principles of the structure, function, and use of systems and methods disclosed herein. One or more examples of these non-limiting embodiments are illustrated in the selected examples disclosed and described in detail with reference made to the figures in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

The systems, apparatuses, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these the apparatuses, devices, systems or methods unless specifically designated as mandatory. In addition, elements illustrated in the figures are not necessarily drawn to scale for simplicity and clarity of illustration. For ease of reading and clarity, certain components, modules, or methods may be described solely in connection with a specific figure. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware.

The term “software” is used expansively to include not only executable code, for example machine-executable or machine-interpretable instructions, but also data structures, data stores and computing instructions stored in any suitable electronic format, including firmware, and embedded software. The terms “information” and “data” are used expansively and includes a wide variety of electronic information, including executable code; content such as text, video data, and audio data, among others; and various codes or flags. The terms “information,” “data,” and “content” are sometimes used interchangeably when permitted by context.

It should be noted that although for clarity and to aid in understanding some examples discussed herein might describe specific features or functions as part of a specific component or module, or as occurring at a specific layer of a computing device (for example, a hardware layer, operating system layer, or application layer), those features or functions may be implemented as part of a different component or module or operated at a different layer of a communication protocol stack. Those of ordinary skill in the art will recognize that the systems, apparatuses, devices, and methods described herein can be applied to, or easily modified for use with, other types of equipment, can use other arrangements of computing systems such as client-server distributed systems, and can use other protocols, or operate at other layers in communication protocol stacks, than are described.

The technologies disclosed herein provide cost-effective solutions to traditional problems associated with asset tracking, particularly in large structures and groups of structures, or across defined areas. The technologies disclosed herein advantageously facilitate the rapid and accurate tracking and locating of physical assets and objects—and in some cases associated objects or persons—within structures or across defined areas such as, for example, campuses. In some embodiments, tracking can be continuous, and can including the tracking and locating of multiple assets.

In general, the current location of an asset can be tracked and logged each time an associated asset tracking device comes in proximity to one or more RFID tags placed at known locations within a structure or a defined area. The asset's location can be logged, for example, when it crosses or enters a threshold, doorway, elevator, hall, or other location in a structure at which one or more of the RFID tags have been placed. Asset information, including identifying information, associated information (e.g., related equipment, persons, and the like), and/or previously-stored location data can be transmitted to a remote server for storage and/or management.

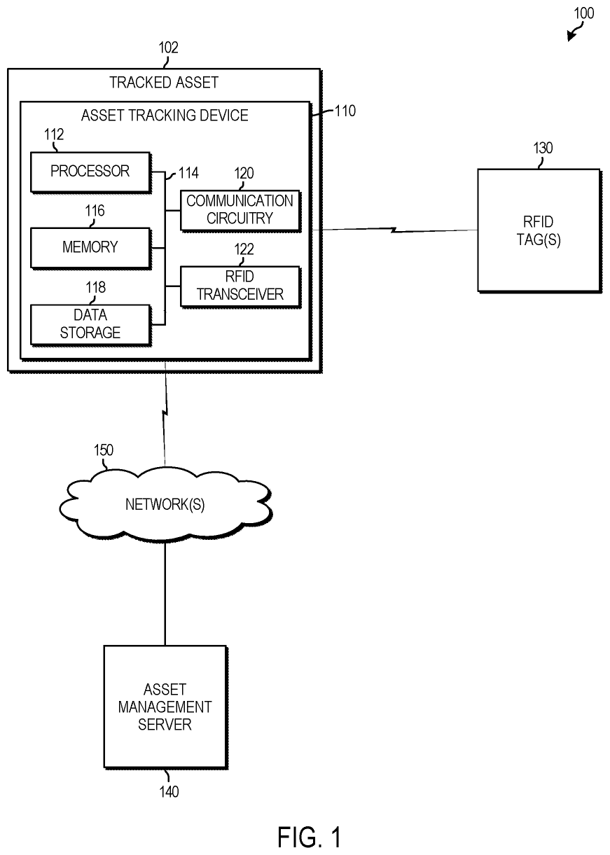

Referring now to FIG. 1, in one embodiment, a system 100 for tracking one or more assets 102 includes one or more RFID tags 130, an asset management server 140, and/or one or more networks 150. The tracked asset(s) 102 can be any type of physical object in a structure or a facility. For example, in some embodiments, the tracked asset(s) 102 can include beds, supply carts, infusion pumps, mobile computers, stands, and any other types of equipment within hospital buildings, across medical campuses, or within any other type of structure. Additionally, each of the tracked assets 102 includes an asset tracking device 110, as discussed in more detail below.

One or more of the RFID tags 130 can be permanently or temporarily attached to or embedded in stationary objects or features of a structure (e.g., commercial building, medical building, installation, etc.) at known locations. For example, one or more RFID tags 130 can be placed in (e.g., attached to, embedded in, etc.) a doorway threshold or frame, an elevator, a hall, a floor, a wall, or any other object or feature of a structure having a known location. In some embodiments, the RFID tags 130 can be installed in an array that can be a linear array or in multiple linear arrays (e.g., on each side of a doorway or threshold). Additionally or alternatively, the RFID tags 130 can be installed in a matrix pattern or any other suitable pattern. When an array or multiple arrays of RFID tags 130 are used, individual RFID tags 130—or groups of RFID tags 130—can be spaced in a pattern that results in only certain, e.g., one RFID tag 130, or a group of the RFID tags 130, being detected by a particular asset tracking device 110 when the associated asset 102 is moved, such as through a doorway.

In some embodiments, each of the RFID tags 130 can be configured to store data indicative of the known location of the object or feature of the structure to which it is attached or embedded (e.g., “Room 202,” “Elevator C1,” “Cardiac Rehab Hall—Location A1,” “Maintenance Shed,” “Supply Closet 4,” or any other suitable information describing a specific location). Additionally or alternatively, each of the RFID tags 130 can include a unique identifier, which can be associated with data or information managed, stored, or otherwise accessible to the asset management server 140. For example, in such embodiments, the unique identifier of each RFID tag 130 can be associated with the known location data of the RFID tag 130 and/or the object or feature to which the RFID tag 130 is attached or embedded. That is, the asset management server 140, or any other computing device in communication therewith, can use the unique identifier of a particular RFID tag 130 to retrieve, from a local or remote storage device, the known location information associated with the RFID tag 130. Varying implementations of RFID tags 130 may also include security features to prevent tampering, which may include configuration as a write-once read-many storage device, encryption or encoding of stored data, or storage of password and/or authentication data that must be verified against data provided by a device reading or writing to the RFID before the stored memory may be modified or read.

In an embodiment, one or more of the RFID tags 130 are passive RFID tags (i.e., unpowered RFID tags). In such embodiments, as discussed herein, the passive RFID tags 130 can be configured to be energized (e.g., remotely powered) in response to the receipt of a specific radio frequency (or a range of radio frequencies) or other signal from an asset tracking device 110 of a tracked asset 102 in close proximity thereto, and to automatically transmit stored data while energized. In varying implementations, RFID tags 130 and asset tracking devices 110 may be capable of such communication at distances between about 2 meters and about 1 meter or less, depending upon characteristics such as signal frequency, antenna configuration, and number of transmitters and receivers. Additionally or alternatively, one or more of the RFID tags 130 are active RFID tags (i.e., powered RFID tags).

As discussed, each of the tracked assets 102 can include an asset tracking device 110 associated therewith (e.g., attached, adhered, joined, embedded, or otherwise connected). The asset tracking device 110 can include, or otherwise be associated with, an RFID transceiver 122 having one or more antennas. In some embodiments, such as the one illustratively shown in FIG. 1, the RFID transceiver 122 can be a component of (e.g., form part of) the asset tracking device 110. In other embodiments, the RFID transceiver 122 can be a component separate from the asset tracking device 110. In any event, the RFID transceiver 122—and/or the asset tracking device 110 including the RFID transceiver 122—can be positioned such that its antenna will pass within a suitable operating range of an RFID tag 130 during operation of the system 100 (e.g., between about 1 inch and about 10 inches, about 1 meter or less, between about 1 meter and about 2 meters, depending upon a particular implementation). These suitable ranges may also be referred to as a reference proximity, and in some implementations may be purposely configured to have a limited reference proximity, such as by using only passive RFID tags, limiting the range of the RFID transceiver, or both. An asset tracking system with a purposefully limited reference proximity is advantageous due to the reduced complexity and operational requirements of the RFID tags and transceivers, as well as improved accuracy and mitigation of unintentional and/or errant signal communications. As an example, where the reference proximity is configured to be around 12 inches or less, the passage of configured assets through particular tracked areas may be determined with high accuracy, and without the risk of RFID or other wireless signals from other devices in nearby rooms, mounted to the ceiling in the same room, or carried in a pocket of a nearby person being undesirably detected.

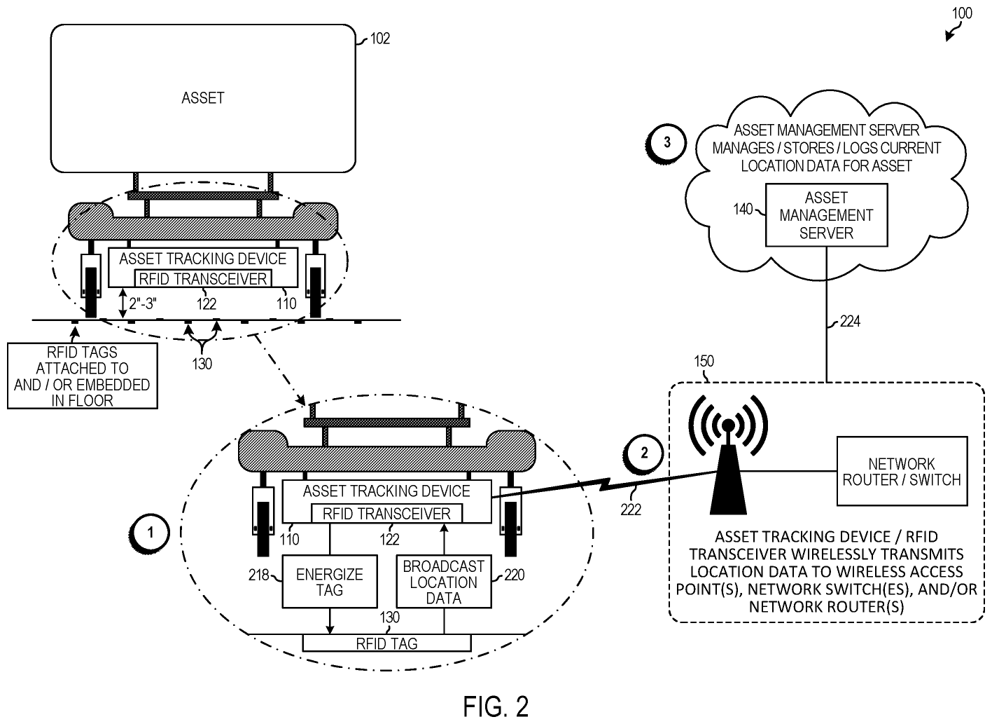

In operation, referring now to FIG. 2, the RFID transceiver 122 is configured to transmit a radio frequency selected to energize one or more RFID tags 130 in proximity thereto (see transmission 218). As discussed in more detail below, the one or more RFID tags 130 are configured to wirelessly transmit or broadcast location data, information associated with the RFID tag 130 (e.g., a unique identifier, etc.), and/or asset-specific data in response to receiving the radio frequency being transmitted by the RFID transceiver 122 (see transmission 220). The RFID transceiver 122 receives the location data and/or information transmitted by the RFID tag 130. Thereafter, the asset tracking device 110 transmits the received data to the asset management server 140 via one or more wireless and/or wired communication links (see wireless transmission 222 and wired and/or wireless transmission 224). In embodiments in which more than one RFID tag 130 is installed at a known location, the asset tracking device 110 can, in some examples, be configured to only transmit the location data, tag information data (e.g., unique identifier, etc.), and/or asset-specific data in response to receiving data from two or more RFID tags 130 (or any threshold number of the RFID tags 130).

Referring back to FIG. 1, the asset tracking device 110 can be embodied as any type of computing device or server capable of processing, communicating, storing, maintaining, and transferring data. For example, the asset tracking device 110 can be embodied as a microcomputer, a minicomputer, a custom chip, an embedded processing device, a mobile computing device, a handheld computer, a smart phone, a tablet computer, a personal digital assistant, a laptop computer, a desktop computer, and/or other computing device or suitable programmable device. In some embodiments, the asset tracking device 110 can be embodied as a computing device integrated with other systems or subsystems. As illustratively shown in FIG. 1, the asset tracking device 110 includes a processor 112, a system bus 114, a memory 116, a data storage 118, communication circuitry 120, and an RFID transceiver 122. Of course, the asset tracking device 110 can include other or additional components, such as those commonly found in a computing device and/or server (e.g., various input/output devices), in other embodiments. Additionally, in some embodiments, one or more of the illustrative components can be incorporated in, or otherwise from a portion of, another component. For example, the memory 116, or portions thereof, can be incorporated in the processor 112 in some embodiments. Furthermore, it should be appreciated that the asset tracking device 110 can include other components, sub-components, and devices commonly found in a computer and/or computing device, which are not illustrated in FIG. 1 for clarity of the description.

The processor 112 can be embodied as any type of processor capable of performing the functions described herein. For example, the processor 112 can be embodied as a single or multi-core processor, a digital signal processor, a microcontroller, a general purpose central processing unit (CPU), a reduced instruction set computer (RISC) processor, a processor having a pipeline, a complex instruction set computer (CISC) processor, an application specific integrated circuit (ASIC), a programmable logic device (PLD), a field programmable gate array (FPGA), or any other type of processor or processing/controlling circuit or controller.

In various configurations, the asset tracking device 110 includes a system bus 114 for interconnecting the various components of the asset tracking device 110. The system bus 114 can be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations with the processor 112, the memory 116, and other components of the asset tracking device 110. In some embodiments, the asset tracking device 110 can be integrated into one or more chips such as a programmable logic device or an application specific integrated circuit (ASIC). In such embodiments, the system bus 114 can form a portion of a system-on-a-chip (SoC) and be incorporated, along with the processor 112, the memory 116, and other components of the asset tracking device 110, on a single integrated circuit chip.

The memory 116 can be embodied as any type of volatile or non-volatile memory or data storage capable of performing the functions described herein. For example, the memory 116 can be embodied as read only memory (ROM), random access memory (RAM), cache memory associated with the processor 112, or other memories such as dynamic RAM (DRAM), static RAM (SRAM), programmable ROM (PROM), electrically erasable PROM (EEPROM), flash memory, a removable memory card or disk, a solid state drive, and so forth. In operation, the memory 116 can store various data and software used during operation of the asset tracking device 110 such as operating systems, applications, programs, libraries, and drivers.

The data storage 118 can be embodied as any type of device or devices configured for short-term or long-term storage of data such as, for example, memory devices and circuits, memory cards, hard disk drives, solid-state drives, or other data storage devices. For example, in some embodiments, the data storage 118 includes storage media such as a storage device that can be configured to have multiple modules, such as magnetic disk drives, floppy drives, tape drives, hard drives, optical drives and media, magneto-optical drives and media, Compact Disc (CD) drives, Compact Disc Read Only Memory (CD-ROM), Compact Disc Recordable (CD-R), Compact Disc Rewriteable (CD-RW), a suitable type of Digital Versatile Disc (DVD) or Blu-Ray disc, and so forth. Storage media such as flash drives, solid state hard drives, redundant array of individual disks (RAID), virtual drives, networked drives and other memory means including storage media on the processor 112, or the memory 116 are also contemplated as storage devices. It should be appreciated that such memory can be internal or external with respect to operation of the disclosed embodiments. It should also be appreciated that certain portions of the processes described herein can be performed using instructions stored on a computer-readable medium or media that direct or otherwise instruct a computer system to perform the process steps. Non-transitory computer-readable media, as used herein, comprises all computer-readable media except for transitory, propagating signals.

The communication circuitry 120 of the asset tracking device 110 may be embodied as any type of communication circuit, device, interface, or collection thereof, capable of enabling communications between the asset tracking device 110, the asset management server 140, and/or any other computing or processing devices communicatively coupled thereto. For example, the communication circuitry 120 may be embodied as one or more network interface controllers (NICs), in some embodiments. The communication circuitry 120 may be configured to use any one or more communication technologies (e.g., wireless or wired communications) and associated protocols (e.g., Ethernet, Wi-Fi®, WiMAX, etc.) to effect such communication. In the illustrative embodiment, the communication circuitry 120 includes a wireless communication interface (e.g., Wi-Fi®, Bluetooth®, NFC, mesh network, etc.) configured to enable communications between the asset tracking device 110, the asset management server 140, and/or any other computing or processing device. Additionally or alternatively, in some embodiments, the communication circuitry 120 includes a wired communication interface (e.g., Ethernet, coaxial communication interface, USB, serial communication interface, parallel communication interface, etc.) configured to enable communications directly between the asset tracking device 110 and the asset management server 140 via a physical communications connection.

In some embodiments, the asset tracking device 110, the asset management server 140, and/or any other computing or processing devices of the system 100, can communicate with each other over one or more networks 150. The network(s) 150 can be embodied as any number of various wired and/or wireless communication networks. For example, the network(s) 150 can be embodied as or otherwise include a local area network (LAN), a wide area network (WAN), a cellular network, or a publicly-accessible, global network such as the Internet. Additionally, the network(s) 150 can include any number of additional devices (e.g., wireless access points, bridges, switches, routers, etc.) to facilitate communication between the computing devices of the system 100.

Additionally, in some embodiments, the asset tracking device 110 can further include one or more peripheral devices (not shown). Such peripheral devices can include any type of peripheral device commonly found in a computing device such as various user interface devices (e.g., a joystick, buttons, controls, a hardware keyboard, a keypad, a gesture or graphical input device, a motion input device, a vibratory device, a computer mouse, a voice recognition unit, etc.), a display and/or a touchscreen interface, additional data storage, speakers, an audio unit, a peripheral communication device, and any other suitable user interface, input/output device, and/or other peripheral device.

In some embodiments, the asset tracking device 110 can be configured to compute information related to the movement of the tracked asset 102 to which it is attached. For example, two or more RFID tags 130 can be spaced and positioned at a known location of a structure. Based on the known spatial positioning of the RFID tags 130, as well as the location data and/or tag-specific information received from RFID tags 130, the asset tracking device 110 can be configured to compute or determine the direction and/or velocity of the tracked asset 102. In some embodiments, the asset tracking device 110 can include an accelerometer, which can be configured to measure and/or compute information related to the movement of the tracked asset 102 to which it is attached. Such measurements and/or information can be used by the asset tracking device 110 to facilitate movement determinations for the tracked asset 102. In some embodiments, the asset tracking device 110 can also be configured to transmit location data, tag information, and/or asset-specific data to the asset management server 140 only when movement of the tracked asset 102 has been detected (or within a configurable reference time threshold from movement detection). It should be appreciated that such approaches facilitate conservation of power and efficiency. Additionally, in some embodiments, the asset tracking device 110 of each tracked asset 102 can be configured to periodically communicate (e.g., transmit a heartbeat signal, short message, etc.) to the asset management server 140 to ensure that the asset tracking device 110 or other components of the system 100 are operational.

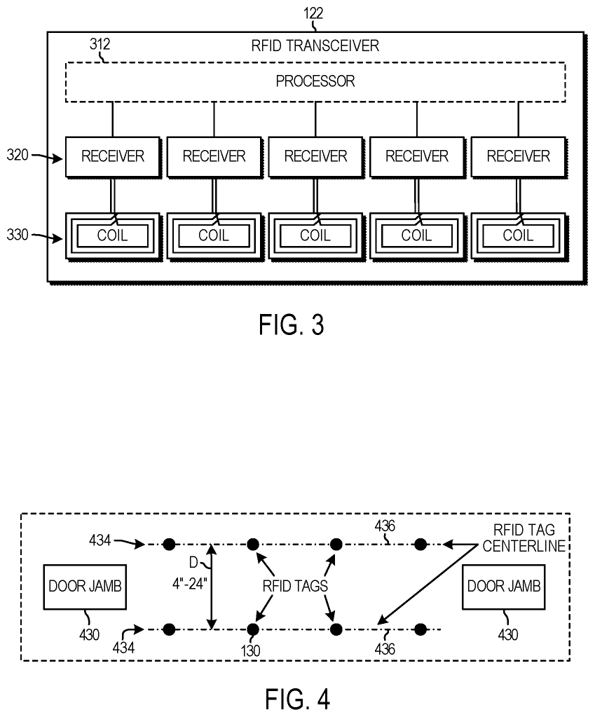

As discussed above, the asset tracking device 110 can include, or otherwise be associated with, an RFID transceiver 122 having one or more antennas. Referring now to FIG. 3, one embodiment of the RFID transceiver 122 is depicted. In such embodiment, the RFID transceiver 122 can include multiple receivers 320, each communicatively coupled with a separate coil 330. In some embodiments, the RFID transceiver 122 can include a processor 312 separate from the processor 112 of the asset tracking device 110.

Referring back to FIG. 1, the asset management server 140 may be embodied as any type of computing device (or devices) capable of performing the functions described herein. As such, the asset management server 140 may include devices and structures commonly found in computing devices such as processors, memory devices, communication circuitry, and data storages, which are not shown in FIG. 1 for clarity of the description. In some embodiments, the asset management server 140 can be a cloud-based server or platform. The asset management server 140 is configured to receive location data, tag data, and/or asset information received from the asset tracking device 110. As discussed above, the asset management server 140 can receive the location data and/or asset information from the asset tracking device 110 via one or more wireless communication links, wired communication links, or combinations thereof. The asset management server 140 can store and manage the location data and/or asset information received from the asset tracking device 110 for each of the tracked assets 102.

In some embodiments, the asset management server 140 can store information associated with or otherwise corresponding to the location data and/or asset information received from the asset tracking devices 110. For example, in some embodiments, the asset management server 140 can store patient identification data (e.g., patient name, patient number, patient social security number, health record data, etc.) in association with data corresponding to a tracked hospital bed (e.g., a tracked asset 102). In such embodiments, the asset management server 140 can be configured to ascertain the location of a patient assigned to that particular bed by cross-referencing the location data and asset data (e.g., an asset identifier, etc.) received from the asset tracking device 110 attached to the hospital bed (e.g., the tracked asset 102) with the stored patient information. It should be appreciated that the asset management server 140 can also be configured to receive and store timestamp data received from the asset tracking device 110 of a tracked asset 102. In such cases, the asset management server 140 can record the received timestamp data in association with the received location data and asset information.

The asset management server 140 can be configured to respond to requests received from computing devices of the system 100. For example, in some embodiments, the asset management server 140 is configured to retrieve and provide information concerning the current location of one of more of the tracked assets 102 in response to a received request. In some embodiments, the asset management server 140 is configured to generate one or more reports relating to the current locations of the tracked assets 102, which can advantageously facilitate inventory and maintenance efforts. Additionally, in some embodiments, the asset management server 140 can be configured to generate one or more alarms or notifications in response to receiving information indicative of a tracked asset 102—or an object or person associated therewith—moving into or out of an approved location or area.

Referring now to FIG. 4, there is shown in plan view a schematic diagram an example RFID tag 130 placement pattern that can be used with the asset tracking system 100. The plan view shows a representative number and spacing of RFID tags 130 in or near a doorway, for example, positioned between the door jambs 430 of the doorway. In an embodiment, one RFID tag 130 can be positioned for receiving the radio frequency transmitted by the RFID transceiver 122. As discussed herein, the RFID tag 130 can be configured to be energized by the received radio frequency signal. In an embodiment, such as the one shown in FIG. 4, a plurality of RFID tags 130 can be placed in a spaced relationship, such as substantially evenly spaced between the door jambs 430. In an embodiment, the spaced relationship can include one or more linear arrays 434 of RFID tags 130, with each linear array 434 including an RFID tag centerline 436. Two or more RFID tag centerlines 436 can be spaced apart a distance D, which can be, for example, between about 4 inches and about 24 inches, or between about 8 inches and about 16 inches, or about 10 inches to about 12 inches. Placement patterns such as that shown in FIG. 4 may be implemented by placement of individual RFID tags 130 by embedding them with a material of the floor, doorjamb 430, or other structure, or by fixing them to a surface or structure with an adhesive or other means of fixture.

In some implementations, placement patterns may be implemented by embedding or otherwise affixing RFID tags 130 to a floor covering that may be positioned in the doorway, or to another structure that may be positioned near a doorway, affixed to a structured of the doorway, or provide part of the structure of a doorway (e.g., such as a part of the door frame itself, including a side jamb, sill, threshold, or piece of casing, for example). This may include, for example, a floor mat or other floor covering, a threshold, or a large adhesive sticker with a predetermined and static pattern of RFID tags 130 affixed thereto, or may include a removable and replaceable portion of vertical door jamb with fully or partially enclosed mounting locations at pre-determined positions for RFID tags 130 such that they are not visible from an exterior of the door jamb, but are still able to receive and transmit signals to and from a transceiver. Floor coverings, vertical door jambs, or other tag mounting structures with fixed patterns of RFID mounting locations and/or integrated or embedded tags may be placed and replaced in their entirety, or may allow for replacement of individual RFID when necessary (e.g., each RFID may be contained within a cavity of the object, and may be removed and replaced if it is damaged or otherwise malfunctions).

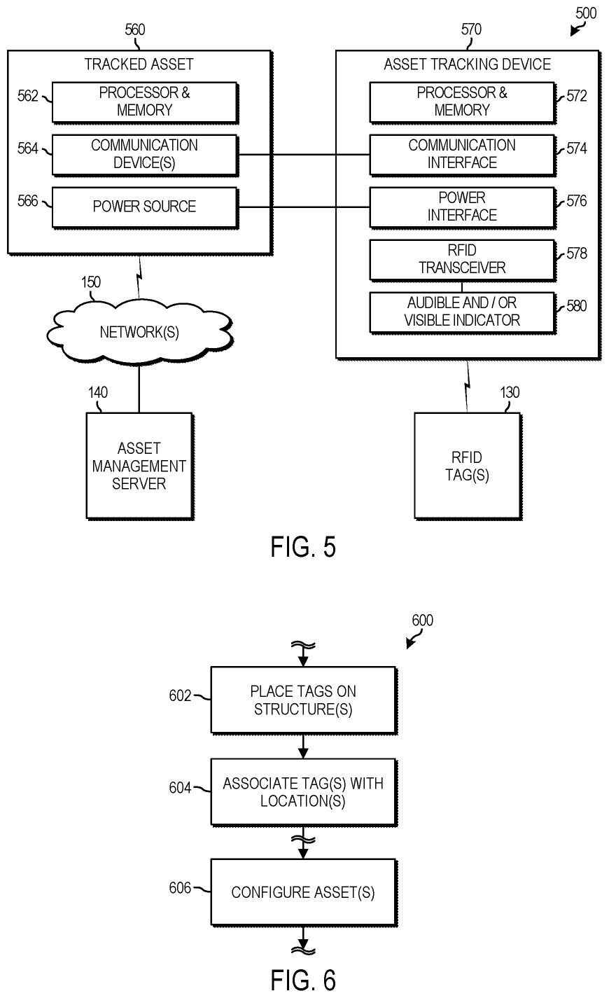

FIG. 5 shows an alternate embodiment of an asset tracking system 500. An asset tracking device 570 is shown which is configured to couple to pre-existing interfaces of a tracked asset 560. The tracked asset 560 includes several integrated components that may exist and function for other purposes separate from the asset tracking device 570, including a processor and memory 562, one or more communication devices 564 (e.g., Wi-Fi, Bluetooth, optical, or cellular data transceivers capable of transmitting and receiving data wirelessly), and a power source 566 (e.g., a hardline power connection suitable for connecting to a wall outlet, a rechargeable or replaceable battery, a combination of hardline power connection and rechargeable battery). As an example, the tracked asset 560 may be a hospital bed having electrical motor functions for raising, lowering, or otherwise adjusting the bed. The bed may have a power source 566 suitable for connecting to an electrical outlet when immobilized, and which includes a rechargeable battery that provides power while the bed is being moved from room to room. The bed may include a Wi-Fi transceiver or other communication device 564 capable of communicating with local Wi-Fi networks or other communication networks. The processor and memory 562 may be configured to provide control signals to electrical motors of the bed, and to receive and transmit data via the communication devices 564.

When coupling the asset tracking device 570 to an asset 560 such as the bed, the tracking device 570 may not include any dedicated wireless communication device itself, and may not include its own dedicated power source. Rather, the tracking device 570 may couple to the power source 566 of the asset 560 via a power interface 576, and may communicatively couple to the communication devices 564 via a communication interface 574. As one example, the asset tracking device 570 may receive power and may exchange data over a USB connection between the tracking device 570 and the asset 560. The asset 560 may be in direct communication with the asset management server 140 over the network 150, while the tracking device 570 is in direct communication with the RFID tags 130 via an RFID transceiver 578 or other reader when in proximity thereto. In this way, the communication device 564 of the tracked asset 560 can act as a communications proxy between the asset tracking device 570 and the asset management server 140 or other devices communicatively coupled to the system 500 via the one or more networks 150.

In addition to the communication interface 574 and power interface 576, the tracking device 570 may include a processor and memory 572, RFID transceiver 578, and an audible and/or visible indicator 580 (e.g., a speaker, an LED indicator, or other indicator) which may be activated in certain scenarios to provide notice to nearby personnel, as will be described in more detail below. Of course, the asset tracking device 570 can include other or additional components, such as those commonly found in a computing device (e.g., various input/output devices). It should be appreciated that by relying on the preexisting capabilities of the tracked asset 560, the design and functionality of the tracking device 570 may be simplified.

FIG. 6 shows an exemplary set of steps 600 that may be performed with an asset tracking device or system (e.g., the asset tracking devices 110, 570, the asset tracking systems 100, 500) to configure an asset (e.g., the asset 102, the asset 560, etc.) for tracking. As tags 130 are associated with locations (e.g., by an installer or user that is at the location and using a handheld computing device to submit tag information to the system), the user may place 602 each associated tag on a static structure at the identified location. As has been described, this may include placing tags on the floor, on a doorjamb or wall, or on other static structures at which tracking capabilities are desired. This may also include placing floor coverings, thresholds, or other objects having integrated and pre-arranged patterns of tags, as has been described.

The system may associate 604 one or more tags 130 with locations in which an asset 102, 560 will be tracked, which may include receiving information from a user device that identifies a unique identifier associated with the tag 130, as well as information that identifies a location at which the tag 130 will be placed. The tag location may be expressed in varying ways. As one example, the location may be another unique identifier that is associated in a database with a description of the location, or it may itself be a description of the location. The location may be defined with varying levels of granularity, and may include or be associated with a particular building, a floor of the building, a region within the building, a particular doorway or hallway within the building, a particular room within the building, and, where multiple tags 130 are positioned in a pattern proximate to each other, a position within such a pattern. In this manner, the system may store and associate the unique identifier of the tag 130 with the location of the tag 130, and upon future queries may identify the location of the tag in response to receiving the unique identifier. It should be understood that one or more of the steps of FIG. 6 may be performed in a different order than the one shown. As an example, all tags 130 may be associated 604 with locations prior to placement 602, or may be associated 604 with locations after placement 602 (e.g., as shown in FIG. 6).

The asset(s) 102, 560 may then be configured 606 for use with the system, which may include installing an asset tracking device 110, 570 such as those described above. Configuration 606 may also include configuring the asset tracking device 110, 570 with a unique identifier or other information that describes the asset 102, 560, communicatively coupling the tracking device 110, 570 to the asset management server 140, receiving and storing information from the asset management server 140 on a local storage device of the asset tracking device 110, 570, and configuring one or more active tracking features to be provided by the asset tracking device 110, 570. Active tracking features may be any automated action that is performed in response to receipt of certain RFID tag signals or combinations of RFID tag signals, in addition to the basic features of location tracking and/or location auditing. As an example, an active tracking feature may include defining one or more rooms of the structure in which the asset 102, 560 should not be located, and when RFID tag signals are received that are associated with that room, triggering an audible or visual alarm state. Active tracking features may be configured by interacting with the asset tracking device 110, 570 itself (e.g., via an interface, or via a wireless connection between the tracking device and a handheld computing device), or by interacting with the asset management server 140, and such configurations may be shared between the tracking device 110, 570 and the server 140 (e.g., rules configured remotely on the server may be pushed to the tracking device, and rules configured at the tracking device may be pushed to the server 140). Tag 130 and location definitions may also be shared similarly, such that the identity and location of a particular tag 130 may be resolved locally by the asset tracking device 110, 570, remotely by the server 140, or both. Similarly, active features may be evaluated and acted upon locally, remotely, or both, as will be described in more detail below.

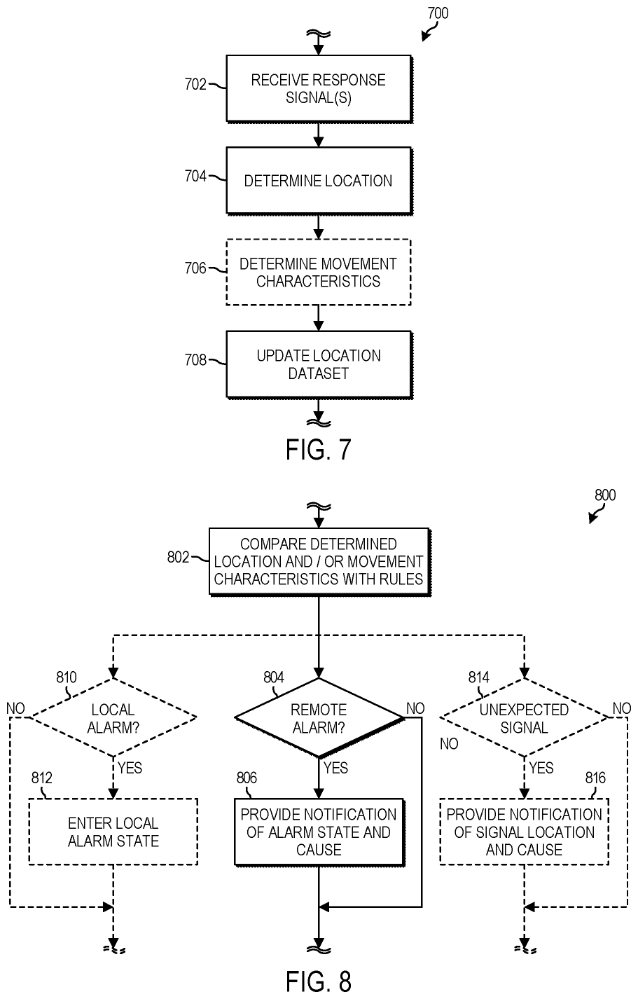

FIG. 7 shows a set of steps 700 that may be performed with an asset tracking system to determine a location of a tracked asset 102, 560, such as that configured 606 during the steps of FIG. 6. Once configured, the asset tracking device 110, 570 may begin to receive 702 response signals from RFID tags 130 within its readable proximity, as has been previously described. As signals are received 702, the system (e.g., the asset tracking device, the server, or both) may determine 704 the identity and location of the tag 130 associated with each received signal and, in some embodiments, may determine 706 one or more movement characteristics of the asset 102, 560 based on the received signals. As one example, where the asset tracking device 110, 570 receives two unique identifiers, A123 and B123, it may provide such information to the server 140, or may search a locally stored dataset, to determine that A123 and B123 are each associated with room 123 of a facility. As an example of determining 706 movement characteristics, the system may examine timestamps associated with the receipt of the two signals to determine that A123 was received moments before B123, and may determine based upon location and pattern descriptions of each tag 130, that A123 is at the outside edge of room 123, while B123 is at the inside edge of room 123. This information may be used to determine that the tracked asset 102, 560 was entering room 123 when it was detected (e.g., the outside edge tag is detected prior to the inside edge tag), and such information may be used to update location records for the tracked asset 102, 560 to indicate presence in room 123. Other movement characteristics that may be determined based upon patterned response signals from RFID tags 130 include the speed at which an asset 102, 560 is moving, the orientation of the asset as it moves through a doorway, into an elevator, or down a hallway, the direction in which an asset moved upon exiting a doorway, and other information.

Based on the determined 704 location and/or determined 706 movement characteristics, the system may update 708 the location dataset. That may include updating 708 the location datasets for one or more assets 102, 560 based upon the received 702 signals, which may include updating stored records that indicate the current and past locations of assets 102, 560. The system may be configured to track the current location and/or movement characteristics of each tracked asset 102, 560, and may display such information in a real-time display or dashboard interface via a web service or other software application. The system may also be configured to audit the locations of particular assets 102, 560 over a period of time for which information is available, and may display such information as a timeline of locations and location changes, or otherwise.

In some implementations, the system may provide one or more reactive tracking features beyond tracking of the asset location. As an example, FIG. 8 shows a set of steps 800 that may be performed with an asset tracking system to provide location and motion based alerts for tracked assets (e.g., the asset 102, the asset 560, etc.). As signals are received 702, the system may begin to compare 802 the determined locations and/or movement characteristics with one or more rules to evaluate whether they are satisfied. This may include evaluating 804 one or more remote rules (e.g., rules being executed and/or evaluated on the server 140) in addition to, or as an alternative to, enforcement of local rules 810. Remotely evaluated rules may include rules such as those restricting an asset to certain areas, which in some implementations may also be evaluated locally by an asset tracking device itself. Remotely evaluated rules may also include rules that may only be evaluated and/or triggered based on information that is available to the server 140 or another remote device, and may not be available locally to the asset tracking device 110, 570. As an example, this may include active tracking features that trigger an alarm state based upon the positions of two or more assets 102, 560, such as where multiple assets are moved to a single area which they may or may not be restricted from, but the system determines that there is no reason for all of those assets to be in the same location (e.g., multiple beds in a room or area of a facility that is generally restricted to a single bed at a time such as an isolation or quarantine area or a private treatment room). Other examples include information from automated cameras, security systems, emergency systems, or other sensors that may generate information that is available to the server 140, but may not be available to the asset tracking device 110, 570 locally. Such information might identify purposeful attempts to bypass the RFID tracking system, or might be used to modify areas where certain assets are allowed (e.g., where a security alert indicates a certain floor is locked down and no assets 102, 560 should be moved to that floor, whether or not they are customarily allowed there, or where a security alert requires that some number of assets 102, 560 be moved to or through an area that they are typically restricted from).

In cases where a remote alarm 804 or rule is triggered, the system may provide 806 one or more notifications or signals indicating the alarm state and the cause or circumstances of the alarm state. Such information may be provided 806 to security personnel or automated security systems, or may be provided to one or more asset tracking devices 110, 570 that are in communication with the server 140. As an example, violation of a remote security rule may cause the server 140 to transmit updated configurations or other information to an asset tracking device 110, 570 that may cause a local alarm state to occur 812, even in the absence of the violation of any locally evaluated rules 810.

While not required, in some implementations the asset tracking device 110, 570 itself may be configured to evaluate locally configured rules 810 to determine if any local alarm conditions are met, and where an alarm condition is met, entering 812 a local alarm state (e.g., activation of the audible and/or visible indicator 180). Evaluating and triggering alarms entirely locally to the asset tracking device 110, 570 may be advantageous in that it eliminates the need communicate with a remote server to determine an alarm state, and so may be performed more quickly and in the absence of reliable communication with the server 140 or another remote device. Local alarms may be configured to trigger based upon a variety of circumstances, such as the asset 102, 560 being present in a location it is not permitted in, or being present outside of a list of permitted locations, or may be based upon movement characteristics, such as the asset 102, 560 moving at an unsafe speed, or being moved through doorways, hallways, or into elevators in an unsafe orientation. There may be some overlap in the type and effect of locally configured 810 and remotely configured 804 rules, which may be advantageous for at least the reasons described above.

While not required, in some implementations, the system may be configured with active tracking features that are designed to detect failures in RFID tags 130, misconfiguration of RFID tags 130, or intentional attempts to bypass the tracking system. These active tracking features may monitor received signals (e.g., step 702 of FIG. 7) for the presence of any unexpected signal characteristics 814, and may provide notifications 816 of the locations and circumstances surrounding any unexpected signal characteristics.

As an example, one rule configured to detect unexpected signals might be triggered when a single tracking device 110, 570 receives signals from two RFID tags 130 that are not proximately located, such that near-simultaneous receipt of the two signals should be impossible, which may indicate misconfiguration or misplacement of RFID tags 130, or intentional attempts to bypass the tracking system.

As another example, one rule may be configured to monitor for groupings of received signals responses from RFID tags 130 that should respond near-simultaneously, and trigger when the expected group of responses is not received, which may indicate that an RFID tag 130 has failed or malfunctioned, or has been tampered with. As further example, this may include where a doorway has a leading and trailing RFID tag 130 (e.g., RFID tags A123 and B123, described above), but only a single response signal is received from an asset 102, 560 moving through the doorway. The above example may also apply where two or more redundant RFID tags 130 having substantially the same location and purpose are placed in order to prevent malfunction of the system due to the failure or dislocation of a single tag 130.

Some of the figures can include a flow diagram. Although such figures can include a particular logic flow, it can be appreciated that the logic flow merely provides an exemplary implementation of the general functionality. Further, the logic flow does not necessarily have to be executed in the order presented unless otherwise indicated. In addition, the logic flow can be implemented by a hardware element, a software element executed by a computer, a firmware element embedded in hardware, or any combination thereof.

Exemplary Combinations

Example 1 includes a system for tracking mobile assets, the system includes: a tracking device mounted to an asset, the tracking device having one or more transceivers; and one or more RFID tags mounted to one or more static structures; wherein: the one or more transceivers of the tracking device energize or trigger one or more of the RFID tags, in response to being energized or triggered, the energized or triggered RFID tags transmit stored identification data, the identification data being associated with predetermined location data, and the one or more transceivers of the tracking device receive or detect the identification data transmitted by the energized or triggered RFID tags.

Example 2 includes the system of Example 1, and wherein the transceiver or an antenna of the transceiver is located near the floor or sides of the asset to facilitate the triggering of a nearby RFID tag.

Example 3 includes the system of Examples 1 and 2, and wherein the tracking device contains an accelerometer to detect the speed and direction of the asset/patient.

Example 4 includes the system of any Examples 1-3, and wherein a plurality of RFID tags is used at each location to allow the tracking device to detect the speed and direction of movement for the asset.

Example 5 includes the system of any one of Examples 1-4, and wherein the tracking device transmits the identification data to a central server to determine and record its current location and movement.

Example 6 includes the system of any one of Examples 1-5, and wherein the central server comprises an alarm system to alert users to assets or patients moving into or out of approved locations or areas.

Example 7 includes the system of any one of Examples 1-6, and wherein the one or more RFID tags include one or more passive RFID tags, active RFID tags, or short-range tags.

Example 8 includes the system of any one of Examples 1-7, and wherein the one or more static structures include one or more thresholds, floors, walls, vertical jambs, or other locations.

Example 9 includes a system for tracking patients in mobile hospital beds, the system including: a tracking device mounted to a mobile hospital bed associated with a patient, the tracking device having one or more transceivers; and one or more RFID tags mounted to one or more static structures; wherein: the one or more transceivers of the tracking device energize or trigger one or more of the RFID tags, in response to being energized or triggered, the energized or triggered RFID tags transmit stored identification data, the identification data being associated with predetermined location data, and the one or more transceivers of the tracking device receive or detect the identification data transmitted by the energized or triggered RFID tags.

Example 10 includes the system of Example 9, and wherein the transceiver or an antenna of the transceiver is located near the floor or sides of the tracking device to facilitate the triggering of a nearby RFID tag.

Example 11 includes the system of any of Examples 9 and 10, and wherein the tracking device contains an accelerometer to detect the speed and direction of the mobile hospital bed.

Example 12 includes the system of any of Examples 9-11, and wherein a plurality of RFID tags is used at each location to allow the tracking device to detect the speed and direction of movement for the mobile hospital bed/patient.

Example 13 includes the system of any of Examples 9-12, and wherein the tracking device transmits the identification data to a central server to determine and record its location and movement.

Example 14 includes the system of any of Examples 9-13, and wherein the central server comprises an alarm system to alert users to mobile hospital beds moving into or out of approved locations or areas.

Example 15 includes the system of any of Examples 9-14, and wherein the one or more RFID tags include one or more passive RFID tags, active RFID tags, or short-range tags.

Example 16 includes the system of any of Examples 9-15, and wherein the one or more static structures include one or more thresholds, floors, walls, vertical jambs, or other locations.

Example 17 includes an asset tracking system including: a server configured to store a tag location dataset, wherein the tag location dataset includes a plurality of tag identifiers that are each associated with a tag location, a plurality of asset locations that are each associated one or more tag locations, and an asset identifier; an asset tracking device in communication with the server, the asset tracking device comprising a passive tag transceiver configured to communicate with passive tags, wherein the asset tracking device is fixed to a wheeled asset; one or more processors configured to: associate a plurality of tags with the tag location dataset by adding tag identifiers to the plurality of tag identifiers, wherein the plurality of tags comprise passive tags fixed to static structures at a tracked facility; receive one or more response signals via the passive tag transceiver in response to an energizing signal provided by the passive tag transceiver; determine one or more responding tag locations that are associated with the one or more response signals based on the tag location dataset; determine a facility location that is associated with the one or more responding tag locations based on the tag location dataset; and update an asset location of the plurality of asset locations based on the facility location, wherein the asset location corresponds to the asset identifier of the wheeled asset.

Example 18 includes the asset tracking system of Example 17, and wherein the one or more processors include at least one processor of the server and at least one processor of the asset tracking device.

Example 19 includes the asset tracking system of any of Examples 17 and 18, and wherein the one or more processors are further configured to: store at least a portion of the tag location dataset on a local memory of the asset tracking device; determine the one or more responding tag locations using a processor of the asset tracking device and the tag location dataset stored on the local memory.

Example 20 includes the asset tracking system of any of Examples 17-19, and wherein the processor of the asset tracking device is further configured to activate a local alarm device of the asset tracking device based on the tag location dataset stored on the local memory and the determined facility location, and provide an audible signal, visible signal, or both indicating an alarm state.

Example 21 includes the asset tracking system of any of Examples 17-20, and wherein the one or more processors are further configured to: identify an unexpected signal within the one or more response signals based on the tag location dataset, wherein the unexpected signal comprises one or several signals received within a period of time that would not occur in the absence of a malfunction or misconfiguration of the plurality of tags; and provide an indication of the unexpected signal that includes the facility location where the unexpected signal occurred, a description of the unexpected signal, and a description of an expected signal that should be received from tags at that facility location.

Example 22 includes the asset tracking system of any of Examples 17-21, and wherein the one or more processors are further configured to determine one or more movement characteristics associated with the wheeled asset based on the one or more responding tag locations.

Example 23 includes the asset tracking system of any of Examples 17-22, and wherein the one or more movement characteristics include one or more of: a direction that the wheeled asset is moving relative to the one or more responding tag locations; a speed at which the wheeled asset is moving; and an orientation of the wheeled asset while moving.

Example 24 includes the asset tracking system of any of Examples 17-23, and wherein the asset tracking device further includes: a communication interface that is configured to allow the asset tracking device to communicate with the server via a pre-existing communication device of the wheeled asset to which the communication interface is communicatively coupled; and a power interface that is configured to provide power to the asset tracking device from a pre-existing power source of the wheeled asset to which the power interface is coupled.

Example 25 includes the asset tracking system of any of Examples 17-24, and wherein the passive tag transceiver is configured to communicate only with tags that are within a reference proximity of the passive tag transceiver, wherein the reference proximity is between about 4 inches and about 12 inches, and wherein the passive tag transceiver fixed to the wheeled asset at a position that places the static structures at the tracked facility within the reference proximity of the passive tag transceiver.

Example 26 includes the asset tracking system of any of Examples 17-25, and wherein the static structures at the tracked facility include floors and door frames.

Example 27 includes a tag mounting structure for use with an asset tracking system, the tag mounting structure includes: a body that includes a plurality of fixture points in a predetermined pattern, wherein the plurality of fixture points are each configured to receive and hold a passive tag at a position in the predetermined pattern; and a plurality of passive tags positioned at the plurality of fixture points, wherein the plurality of passive tags are configured to provide a response signal in response to being energized by a passive tag transmitter.

Example 28 includes the tag mounting structure of Example 27, and wherein the tag mounting structure includes one or more of a floor covering, a wall, a vertical door jamb, and a door threshold.

Example 29 includes a tracked asset including: an asset structure including a set of wheels that allow the tracked asset to be moved; an asset tracking device fixed to the asset structure, the asset tracking device comprising a passive tag transceiver configured to communicate with passive tags and a processor, wherein: the passive tag transceiver is configured to communicate only with tags that are within a reference proximity of the passive tag transceiver, wherein the reference proximity is between about 4 inches and about 12 inches; the passive tag transceiver fixed to the asset structure at a position that places one or more static structures at a tracked facility within the reference proximity of the passive tag transceiver; wherein a plurality of tags are positioned on the one or more static structures at the tracked facility; wherein the processor is configured to: receive one or more response signals via the passive tag transceiver in response to an energizing signal provided by the passive tag transceiver; access a tag location dataset, wherein the tag location dataset includes a plurality of tag identifiers that are each associated with a tag location, an asset location that is associated with one or more tag locations, and an asset identifier associated with the tracked asset; determine one or more responding tag locations that are associated with the one or more response signals based on the tag location dataset; determine a facility location that is associated with the one or more responding tag locations based on the tag location dataset; and update the asset location based on the facility location, wherein the asset location corresponds to the asset identifier of the tracked asset.

Example 30 includes a method for tracking wheeled assets within a building, the method including: coupling an asset tracking device to a wheeled asset, wherein the asset tracking device comprises a passive tag transceiver configured to communicate with passive tags that are located within a reference proximity of the passive tag transceiver; storing a unique tag identifier on each of a plurality of passive tags; storing a tag location dataset on a server, wherein the tag location dataset comprises a plurality of tag locations that are each associated with unique tag identifiers; fixing each of the plurality of passive tags to static structures at different locations within the building, at positions on those static structures that will place that passive tag within the reference proximity of the passive tag transceiver when the wheeled asset passes through those locations; receiving, with the passive tag transceiver, a plurality of responses from passive tags as the wheeled asset moves within the building, wherein the plurality of responses comprise the unique tag identifiers of the responding passive tags; providing, via a communication device of the asset tracking device, the plurality of responses to the server; determining, with a processor of the server, a location of the wheeled asset based on the plurality of responses and the tag location dataset; and storing, with the processor, the location of the wheeled asset.

Example 31 includes a system for tracking assets according to one or more of the inventive principles as shown and described herein.

Example 32 includes an asset tracking device according to one or more of the inventive principles as shown and described herein.

Example 33 includes an asset management server according to one or more of the inventive principles as shown and described herein.

Example 34 includes a tracked asset according to one or more of the inventive principles as shown and described herein.

Example 35 includes a computer-implemented method for tracking assets according to one or more of the inventive principles as shown and described herein.

Example 36 includes one or more machine-readable media having a plurality of instructions stored thereon that in response to being executed by one or more processors cause a computing device to perform the operations substantially as described herein.

Example 37 includes one or more machine-readable media having a plurality of instructions stored thereon that in response to being executed by one or more processors cause an asset tracking device to perform the operations substantially as described herein.

Example 38 includes one or more machine-readable media having a plurality of instructions stored thereon that in response to being executed by one or more processors cause an asset management server to perform the operations substantially as described herein.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto

1. A system for tracking mobile assets, the system comprising:
a tracking device mounted to an asset, the tracking device having one or more transceivers; and
one or more RFID tags mounted to one or more static structures; wherein:
the one or more transceivers of the tracking device energize or trigger one or more of the RFID tags,
in response to being energized or triggered, the energized or triggered RFID tags transmit stored identification data, the identification data being associated with predetermined location data, and
the one or more transceivers of the tracking device receive or detect the identification data transmitted by the energized or triggered RFID tags.

2. The system of claim 1, wherein the transceiver or an antenna of the transceiver is located near the floor or sides of the asset to facilitate the triggering of a nearby RFID tag.

3. The system of claim 1, wherein the tracking device contains an accelerometer to detect the speed and direction of the asset.

4. The system of claim 1, wherein a plurality of RFID tags are mounted to each of the one or more static structures to allow the tracking device to detect the speed and direction of movement for the asset.

5. The system of claim 1, wherein the tracking device transmits the identification data to a central server to determine and record its current location and movement.

6. The system of claim 5, wherein the central server comprises an alarm system to alert users to assets moving into or out of approved locations or areas.

7. The system of claim 1, wherein the one or more RFID tags include one or more passive RFID tags, active RFID tags, or short-range tags.

8. The system of claim 1, wherein the one or more static structures include one or more thresholds, floors, walls, vertical jambs, or other locations.

9-16. (canceled)

17. An asset tracking system comprising:
a server configured to store a tag location dataset, wherein the tag location dataset includes a plurality of tag identifiers that are each associated with a tag location, a plurality of asset locations that are each associated one or more tag locations, and an asset identifier;
an asset tracking device in communication with the server, the asset tracking device comprising a passive tag transceiver configured to communicate with passive tags, wherein the asset tracking device is fixed to a wheeled asset;
one or more processors configured to:
associate a plurality of tags with the tag location dataset by adding tag identifiers to the plurality of tag identifiers, wherein the plurality of tags comprise passive tags fixed to static structures at a tracked facility;
receive one or more response signals via the passive tag transceiver in response to an energizing signal provided by the passive tag transceiver;
determine one or more responding tag locations that are associated with the one or more response signals based on the tag location dataset;
determine a facility location that is associated with the one or more responding tag locations based on the tag location dataset; and
update an asset location of the plurality of asset locations based on the facility location, wherein the asset location corresponds to the asset identifier of the wheeled asset.

18. The asset tracking system of claim 17, wherein the one or more processors include at least one processor of the server and at least one processor of the asset tracking device.

19. The asset tracking system of claim 17, wherein the one or more processors are further configured to:
store at least a portion of the tag location dataset on a local memory of the asset tracking device;
determine the one or more responding tag locations using a processor of the asset tracking device and the tag location dataset stored on the local memory.

20. The asset tracking system of claim 19, wherein the processor of the asset tracking device is further configured to activate a local alarm device of the asset tracking device based on the tag location dataset stored on the local memory and the determined facility location, and provide an audible signal, visible signal, or both indicating an alarm state.

21. The asset tracking system of claim 17, wherein the one or more processors are further configured to:
identify an unexpected signal within the one or more response signals based on the tag location dataset, wherein the unexpected signal comprises one or several signals received within a period of time that would not occur in the absence of a malfunction or misconfiguration of the plurality of tags; and
provide an indication of the unexpected signal that includes the facility location where the unexpected signal occurred, a description of the unexpected signal, and a description of an expected signal that should be received from tags at that facility location.

22. The asset tracking system of claim 17, wherein the one or more processors are further configured to determine one or more movement characteristics associated with the wheeled asset based on the one or more responding tag locations.

23. The asset tracking system of claim 22, wherein the one or more movement characteristics include one or more of:
a direction that the wheeled asset is moving relative to the one or more responding tag locations;
a speed at which the wheeled asset is moving; and
an orientation of the wheeled asset while moving.

24. The asset tracking system of claim 17, wherein the asset tracking device further includes:
a communication interface that is configured to allow the asset tracking device to communicate with the server via a pre-existing communication device of the wheeled asset to which the communication interface is communicatively coupled; and
a power interface that is configured to provide power to the asset tracking device from a pre-existing power source of the wheeled asset to which the power interface is coupled.

25. The asset tracking system of claim 17, wherein the passive tag transceiver is configured to communicate only with tags that are within a reference proximity of the passive tag transceiver, wherein the reference proximity is between about 4 inches and about 12 inches, and wherein the passive tag transceiver fixed to the wheeled asset at a position that places the static structures at the tracked facility within the reference proximity of the passive tag transceiver.

26. The asset tracking system of claim 17, wherein the static structures at the tracked facility include floors and door frames.

27-28. (canceled)

29. A tracked asset comprising:
an asset structure including a set of wheels that allow the tracked asset to be moved;
an asset tracking device fixed to the asset structure, the asset tracking device comprising a passive tag transceiver configured to communicate with passive tags and a processor, wherein:
the passive tag transceiver is configured to communicate only with tags that are within a reference proximity of the passive tag transceiver, wherein the reference proximity is between about 4 inches and about 12 inches;
the passive tag transceiver fixed to the asset structure at a position that places one or more static structures at a tracked facility within the reference proximity of the passive tag transceiver;
wherein a plurality of tags are positioned on the one or more static structures at the tracked facility;
wherein the processor is configured to:
receive one or more response signals via the passive tag transceiver in response to an energizing signal provided by the passive tag transceiver;
access a tag location dataset, wherein the tag location dataset includes a plurality of tag identifiers that are each associated with a tag location, an asset location that is associated with one or more tag locations, and an asset identifier associated with the tracked asset;
determine one or more responding tag locations that are associated with the one or more response signals based on the tag location dataset;
determine a facility location that is associated with the one or more responding tag locations based on the tag location dataset; and
update the asset location based on the facility location, wherein the asset location corresponds to the asset identifier of the tracked asset.

30. (canceled)

31. The system of claim 1, wherein the asset is mobile hospital bed.
